(19) United States
(12) Patent Application Publication
(10) Pub. No.: US 2018/0236536 A1
(43) Pub. Date: Aug. 23, 2018

(54) METHOD OF PRODUCING PLATE-SHAPED SILVER NANOPARTICLES

(71) Applicants: TOYOTA JIDOSHA KABUSHIKI KAISHA, Toyota-shi (JP); KONAN GAKUEN, Kobe-shi (JP)

(72) Inventors: Junya MURAI, Nisshin-shi (JP); Hiroshi YANAGIMOTO, Miyoshi-shi (JP); Kensuke AKAMATSU, Nagaokakyo-shi (JP)

(73) Assignees: TOYOTA JIDOSHA KABUSHIKI KAISHA, Toyota-shi (JP); KONAN GAKUEN, Kobe-shi (JP)

(21) Appl. No.: 15/899,643

(22) Filed: Feb. 20, 2018

(30) Foreign Application Priority Data
Feb. 22, 2017 (JP) 2017-031264

(51) Int. Cl. B22F 1/00; B22F 9/24; C08L 39/06; C07C 47/04; C07C 55/06; C07C 55/22; C07D 307/33; C01B 15/01

(52) U.S. Cl. CPC B22F 1/0018; B22F 9/24; C08L 39/06; C07C 47/04; C07C 55/06; B22F 2301/255; C07D 307/33; C01B 15/01; B22F 2001/0033; B22F 2202/11; C07C 55/22

(57) ABSTRACT

A method of producing plate-shaped silver nanoparticles includes: preparing a mixed liquid by adding, to a solution containing silver ions, a reducing agent having a standard electrode potential within a range from 0.03 V to 0.8 V and polyvinylpyrrolidone having a weight-average molecular weight of 10000 to 40000; and precipitating silver from the silver ions by irradiating the mixed liquid with a microwave.

### INCORPORATION BY REFERENCE

The disclosure of Japanese Patent Application No. 2017-031264 filed on Feb. 22, 2017 including the specification, drawings and abstract is incorporated herein by reference in its entirety.

### BACKGROUND

### 1. Technical Field

The disclosure relates to a method of producing plate-shaped silver nanoparticles from a solution containing silver ions.

### 2. Description of Related Art

A paint made from silver nanoparticles has high brightness and is highly transparent to electromagnetic waves. Such a paint is therefore applied to components that are both functional and aesthetically designed, such as an emblem transparent to millimeter waves.

Examples of a technique for producing silver nanoparticles include a method of producing plate-shaped silver nanoparticles (silver nanoplates) described in Japanese Patent No. 5960374. According to this method, first, an aqueous solution of silver nitrate is added to an aqueous solution containing a polystyrene sulfonic acid and a citric acid to prepare a suspension containing silver seed particles. Next, while an ascorbic acid and silver nitrate are added to the prepared suspension, silver is grown on the silver seed particles. In this way, plate-shaped silver nanoparticles are produced.

### SUMMARY

Japanese Patent No. 5960374 describes the method of producing plate-shaped silver nanoparticles by growing silver on silver seed particles. According to this method, silver seed particles are prepared first, and then silver is grown on the silver seed particles. Thus, it takes about 100 hours to generate plate-shaped silver nanoparticles.

It takes such a long time to generate plate-shaped silver nanoparticles for the following reason. Silver is grown through crystal growth of crystals of the silver seed particles. Hence, it is necessary to reduce silver ions into silver and then grow the silver slowly over time.

Even if the suspension containing the silver seed particles is just heated by a heater or the like in order to promote the growth of silver, the silver may fail to be grown anisotropically on the silver seed particles. Thus, it is difficult to accurately generate plate-shaped silver nanoparticles.

The disclosure provides a method of producing silver nanoparticles, the method making it possible to produce plate-shaped silver nanoparticles accurately within a short period of time.

An aspect of the disclosure relates to a method of producing plate-shaped silver nanoparticles. The method includes: preparing a mixed liquid by adding, to a solution containing silver ions, a reducing agent having a standard electrode potential within a range from 0.03 V to 0.8 V and polyvinylpyrrolidone having a weight-average molecular weight of 10000 to 40000; and precipitating silver from the silver ions by irradiating the mixed liquid with a microwave.

According to the above aspect of the disclosure, the polyvinylpyrrolidone having a weight-average molecular weight of 10000 to 40000 is used as a polymer adsorbent. Thus, the polymer adsorbent is adsorbed onto the precipitated silver so as to inhibit the silver from isotropically growing. Further, the reducing agent having a standard electrode potential within the range from 0.03 V to 0.8 V is used, and thus the silver can be anisotropically grown with the polymer adsorbent adsorbed onto the precipitated silver. In this case, a reduction reaction of the silver can be promoted through irradiation of microwaves. As a result, it is possible to produce plate-shaped silver nanoparticles accurately within a short period of time.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a schematic sectional view of a microwave synthesizer used in an embodiment of the disclosure;

FIG. 2 is a graph illustrating the results of measurement of a reduction rate of silver, which were obtained 10 seconds and 20 seconds after the temperature of a mixed liquid according to Example 1 had reached 130° C.;

FIG. 3A is a photograph of silver nanoparticles according to Example 1;

FIG. 3B is a photograph of silver nanoparticles according to Example 2;

FIG. 3C is a photograph of silver nanoparticles according to Example 3;

FIG. 3D is a photograph of silver nanoparticles according to Example 4;

FIG. 4A is a photograph of silver nanoparticles according to Comparative Example 1;

FIG. 4B is a photograph of silver nanoparticles according to Comparative Example 2;

FIG. 4C is a photograph of silver nanoparticles according to Comparative Example 3; and

FIG. 4D is a photograph of silver nanoparticles according to Comparative Example 4.

### DETAILED DESCRIPTION OF EMBODIMENTS

Hereinafter, a method of producing plate-shaped silver nanoparticles according to an embodiment of the disclosure will be described.

In the present embodiment, first, a solution containing silver ions is prepared. Specifically, an inorganic silver salt that is ionizable in a solvent is prepared, and the inorganic silver salt is ionized in the solvent to generate silver ions. For example, when water is used as the solvent, the inorganic silver salt may be silver nitrate, silver cyanide, or silver acetate. From the viewpoint of, for example, easy availability and chemical stability, silver nitrate is preferably used.

Next, a reducing agent for reducing silver ions into silver and a polymer adsorbent to be adsorbed onto the reduced silver are prepared. The reducing agent and the polymer adsorbent are to be added to the solution containing silver ions. Specifically, the reducing agent to be prepared is a reducing agent having a standard electrode potential within a range from 0.03 V to 0.8 V. A reducing agent having a standard electrode potential within this range can cause silver to anisotropically grow with the polymer adsorbent (described later) adsorbed on precipitated silver.

If the standard electrode potential of a reducing agent is lower than 0.03 V, a precipitation reaction proceeds too rapidly. Hence, precipitation of silver proceeds before the polymer adsorbent is adsorbed onto the precipitated silver. As a result, plate-shaped silver nanoparticles cannot be obtained. On the other hand, the standard electrode potential of silver is 0.8 V, and thus an agent having a higher standard electrode potential than that of silver does not function as a reducing agent, so that silver cannot be precipitated.

Examples of a reducing agent having a standard electrode potential within the range from 0.03 V to 0.8 V include a citric acid (0.03 V), formalin (0.056 V), an ascorbic acid (0.06 V), an oxalic acid (0.49 V), and hydrogen peroxide (0.68 V). Note that numerical values in parentheses are standard electrode potentials of the corresponding substances.

As the polymer adsorbent, polyvinylpyrrolidone (a polyvinylpyrrolidone copolymer) having a weight-average molecular weight of 10000 to 40000 is prepared. Thus, the polyvinylpyrrolidone having a weight-average molecular weight within this range is adsorbed onto silver in a specific direction of the silver, so that growth of the silver in the specific direction is inhibited. As a result, the silver is anisotropically grown through irradiation of microwaves (described later), whereby plate-shaped silver nanoparticles are generated. The polyvinylpyrrolidone having a weight-average molecular weight within this range may be prepared, for example, through generally known graft polymerization.

If the weight-average molecular weight of polyvinylpyrrolidone is lower than 10000, the weight-average molecular weight of the polyvinylpyrrolidone is too low. Hence, the polymer adsorbent fails to be adsorbed onto the silver in a specific direction, and fails to protect the periphery of the silver. As a result, the silver cannot be anisotropically grown through irradiation of microwaves (described later), so that spherical silver nanoparticles are unwantedly generated.

On the other hand, if the weight-average molecular weight of polyvinylpyrrolidone exceeds 40000, the weight-average molecular weight of the polyvinylpyrrolidone is too high. Hence, the polymer adsorbent cannot be adsorbed onto the periphery of each silver particle in an appropriate direction. As a result, the polymer adsorbent agglomerates through irradiation of microwaves (described later), and spherical or polyhedral silver nanoparticles are unwantedly generated.

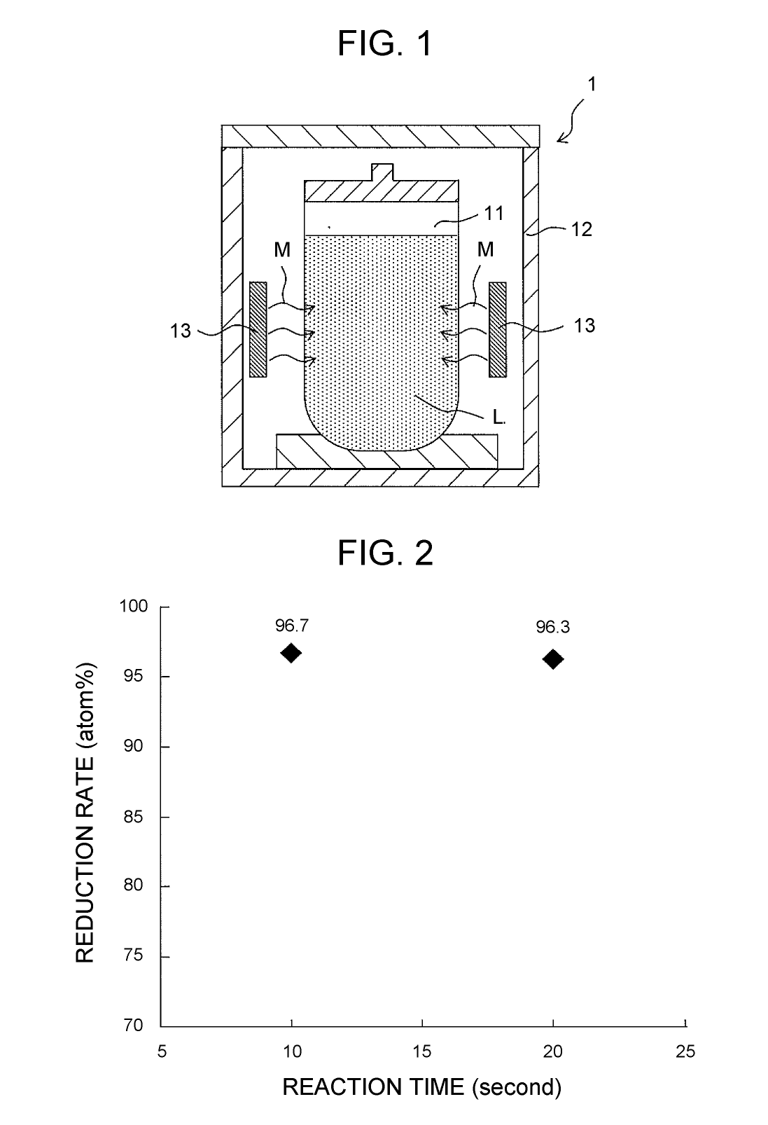

Next, the reducing agent and the polymer adsorbent are added to and mixed with the solution containing silver ions prepared as described, whereby a mixed liquid L is prepared. The thus prepared mixed liquid L is introduced into a microwave synthesizer 1 illustrated in FIG. 1. Specifically, the mixed liquid L is introduced into a container 11 transparent to microwaves M, and the mixed liquid L is irradiated with microwaves M generated by microwave generators 13 disposed in a housing 12. In this way, while silver is precipitated from silver ions, plate-shaped silver nanoparticles are produced.

As long as plate-shaped silver nanoparticles can be generated while silver is precipitated from silver ions, for example, the frequency and the output power of the microwaves are not particularly limited and may be experimentally set based on, for example, the amount of the mixed liquid and the amount of the polymer adsorbent.

In this way, the polymer adsorbent (polyvinylpyrrolidone) is adsorbed onto the precipitated silver to inhibit the silver from isotropically growing, and the silver can be anisotropically grown with the polymer adsorbent adsorbed onto the precipitated silver. Further, the reduction reaction of the silver can be promoted through irradiation of microwaves. As a result, it is possible to produce plate-shaped silver nanoparticles accurately within a short period of time.

The thickness of the thus obtained silver nanoparticle is 1 nm to 50 nm, and the diameter of the surface of the silver nanoparticle seen in a direction perpendicular to a direction in which the silver extends is 10 nm to 500 nm. Note that the diameter of the surface of the silver nanoparticle is a value of the diameter that is obtained when the surface area of the surface is converted into an area of a circle.

Examples of the disclosure will be described below.

### Example 1

A mixed liquid was prepared by mixing, with water used as a solvent, silver nitrate and an ascorbic acid such that the silver nitrate concentration was 10 mM and the ascorbic acid concentration was 20 M. The ascorbic acid is a reducing agent for reducing silver ions into silver. The standard electrode potential of the ascorbic acid is 0.06 V.

Next, polyvinylpyrrolidone (PVP) was further added to the mixed liquid such that the polyvinylpyrrolidone concentration was 20 mM (in terms of unit molecular weight). Polyvinylpyrrolidone is a polymer adsorbent that is adsorbed onto the reduced silver. Note that the polyvinylpyrrolidone used herein is polyvinylpyrrolidone prepared through graft polymerization so as to have a weight-average molecular weight of 10000 (manufactured by Tokyo Chemical Industry Co., Ltd.)

The thus obtained mixed liquid was irradiated with microwaves having a frequency of 2.45 GHz to heat the mixed liquid at 130° C. for 10 minutes. In this way, silver nanoparticles were produced. In this case, in order to check a reduction reaction speed of silver, a reduction rate (atom %) of silver was measured by emission spectral analysis (ICP) 10 seconds and 20 seconds after the temperature of the mixed liquid had reached 130° C. The results are illustrated in FIG. 2.

### Example 2

Silver nanoparticles were produced in a manner similar to that in Example 1. Example 2 differs from Example 1 in that polyvinylpyrrolidone prepared through graft polymerization so as to have a weight-average molecular weight of 40000 (PVP: manufactured by Tokyo Chemical Industry Co., Ltd.) was used as illustrated in Table 1.

### Example 3

Silver nanoparticles were produced in a manner similar to that in Example 1. Example 3 differs from Example 1 in that sodium citrate was used in place of an ascorbic acid as illustrated in Table 1, and the sodium citrate concentration in the mixed liquid was 20 mM. The standard electrode potential of the citric acid is 0.03 V.

### Example 4

Silver nanoparticles were produced in a manner similar to that in Example 1. Example 4 differs from Example 1 in that an oxalic acid was used in place of an ascorbic acid as illustrated in Table 1, and the oxalic acid concentration in the mixed liquid was 20 mM. The standard electrode potential of the oxalic acid is 0.49 V.

### Comparative Example 1

Silver nanoparticles were produced by dissolving 10 mM of silver nitrate and 20 mM of polyvinylpyrrolidone in ethylene glycol used as a solvent, and heating the mixture through irradiation of microwaves in a manner similar to that in Example 1. The standard electrode potential of ethylene glycol is −0.1 V.

### Comparative Example 2

Silver nanoparticles were produced in a manner similar to that in Example 1. Comparative Example 2 differs from Example 1 in that sodium citrate was used in place of an ascorbic acid and polyvinylpyrrolidone (PVP) as illustrated in Table 1, and the sodium citrate concentration in the mixed liquid was 20 mM. Note that the weight-average molecular weight of sodium citrate is 258.

### Comparative Example 3

Silver nanoparticles were produced in a manner similar to that in Example 1. Comparative Example 3 differs from Example 1 in that sodium citrate was used in place of polyvinylpyrrolidone (PVP) as illustrated in Table 1, and the sodium citrate concentration in the mixed liquid was 6 mM. Note that the weight-average molecular weight of sodium citrate is 258.

### Comparative Example 4

Silver nanoparticles were produced in a manner similar to that in Example 1. Comparative Example 4 differs from Example 1 in that polyvinylpyrrolidone (PVP) prepared through graft polymerization so as to have a weight-average molecular weight of 360000 was used as illustrated in Table 1.

TABLE 1

| | | STANDARD | | | |
|---|---|---|---|---|---|
| | | ELECTRODE | | WEIGHT- | |
| | KIND OF | POTENTIAL | KIND OF | AVERAGE | |
| | REDUCING | OF REDUCING | POLYMER | MOLECULAR | MICROWAVE |
| | AGENT | AGENT | ADSORBENT | WEIGHT | IRRADIATION |
| EXAMPLE 1 | ASCORBIC | 0.06 | PVP | 10000 | performed |
| | ACID | | | | |
| EXAMPLE 2 | ASCORBIC | 0.06 | PVP | 40000 | performed |
| | ACID | | | | |
| EXAMPLE 3 | SODIUM | 0.03 | PVP | 10000 | performed |
| | CITRATE | | | | |
| EXAMPLE 4 | OXALIC | 0.49 | PVP | 10000 | performed |
| | ACID | | | | |
| COMPARATIVE | ETHYLENE | −0.1 | PVP | 10000 | performed |
| EXAMPLE 1 | GLYCOL | | | | |
| COMPARATIVE | SODIUM | 0.03 | SODIUM | 258 | performed |
| EXAMPLE 2 | CITRATE | | CITRATE | | |
| COMPARATIVE | ASCORBIC | 0.06 | SODIUM | 258 | performed |
| EXAMPLE 3 | ACID | | CITRATE | | |
| COMPARATIVE | ASCORBIC | 0.06 | PVP | 360000 | performed |
| EXAMPLE 4 | ACID | | | | |

Observation of Appearance of Silver Nanoparticles

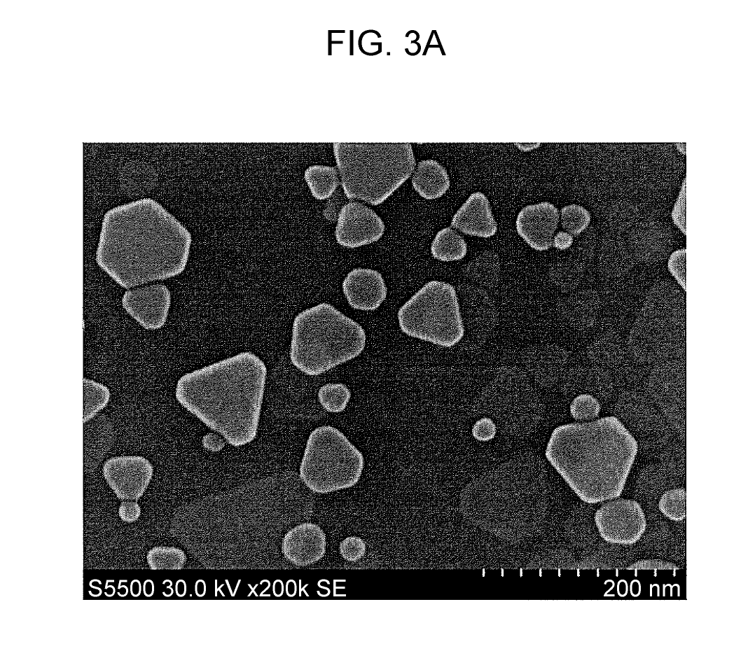

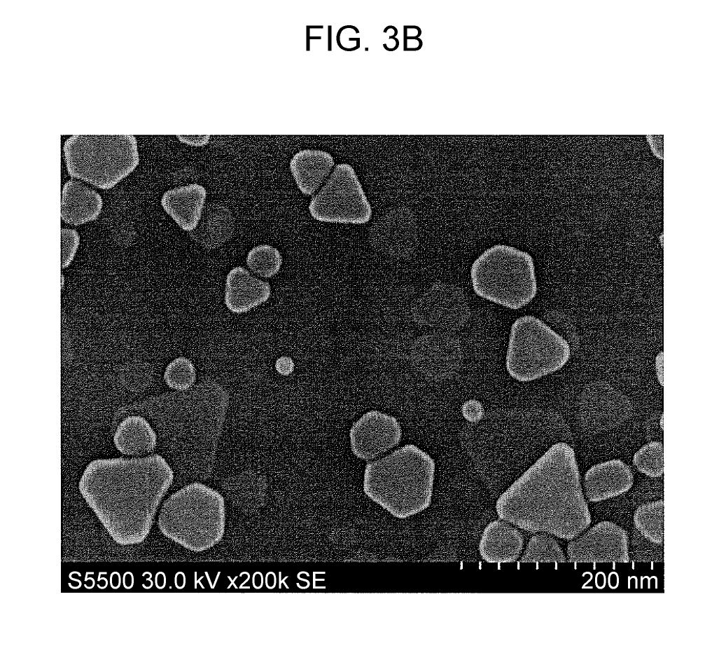

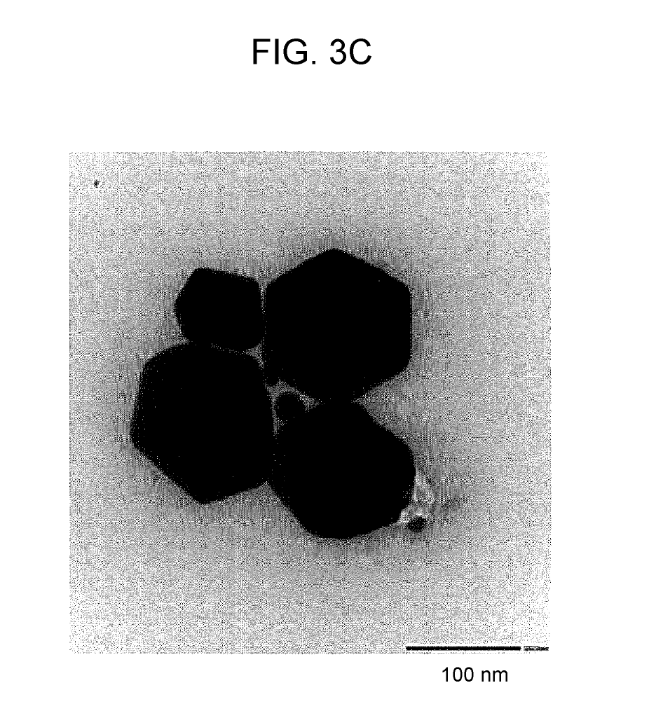

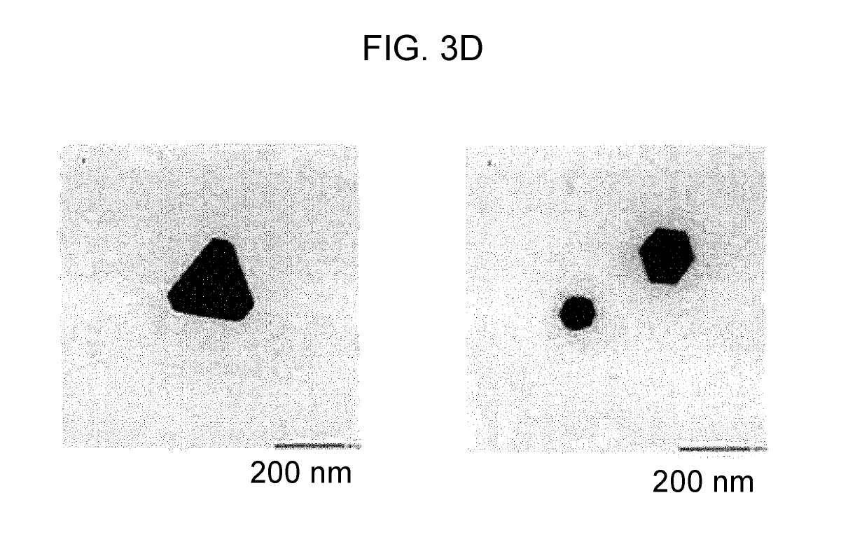

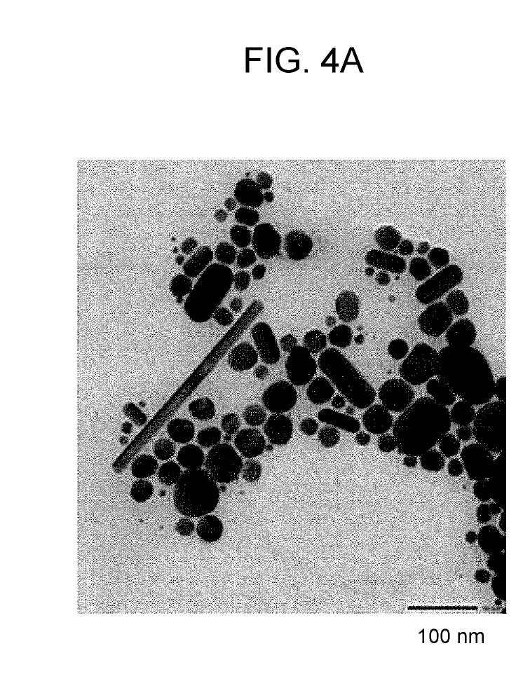

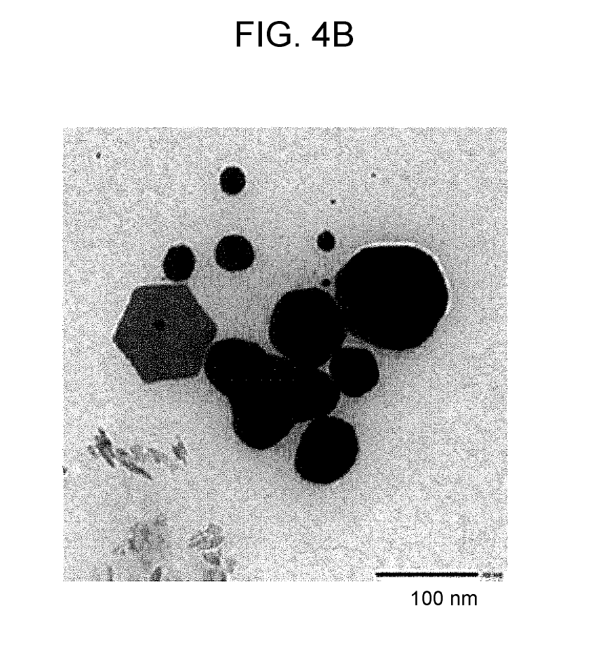

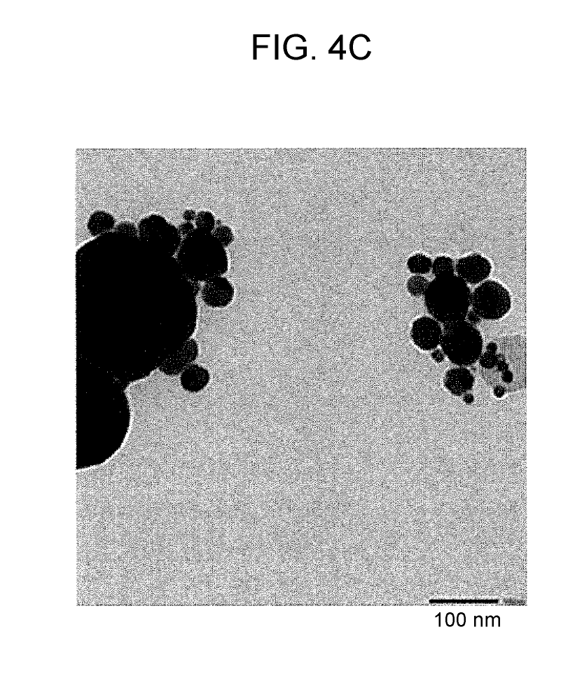

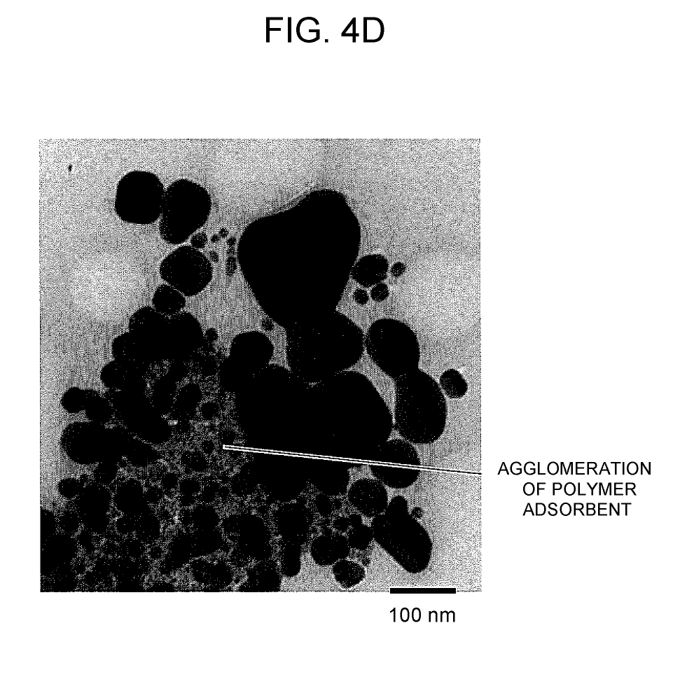

The silver nanoparticles generated in Examples 1 to 4 and Comparative Examples 1 to 4 were observed with the use of a transmission electron microscope (TEM). The results of observation are illustrated in FIG. 3A to FIG. 3D and FIG. 4A to FIG. 4D. FIG. 3A to FIG. 3D are photographs of the silver nanoparticles obtained respectively in Examples 1 to 4, and FIG. 4A to FIG. 4D are photographs of the silver nanoparticles obtained respectively in Comparative Examples 1 to 4.

Result 1 and Consideration 1

As illustrated in FIG. 2, the reduction reaction of silver was substantially completed about 10 seconds after the temperature of the mixed liquid had reached 130° C. through irradiation of microwaves in Example 1. When measurement was performed in a similar manner also in Examples 2 to 4 and Comparative Examples 1 to 4, the reduction reaction of silver was substantially completed about 10 seconds to 1 minute after the temperature of the mixed liquid had reached 130° C. through irradiation of microwaves. This is considered to be because energy is locally applied to the silver ions due to the use of microwaves, so that the reduction reaction is promoted.

Result 2 and Consideration 2

Plate-shaped silver nanoparticles were produced in Examples 1 to 4 as illustrated in FIG. 3A to FIG. 3D, whereas spherical or polyhedral silver nanoparticles were produced in Comparative Examples 1 to 4 as illustrated in FIG. 4A to FIG. 4D.

It is considered that, in Examples 1 to 4, plate-shaped silver nanoparticles were produced because the reducing agents having a low standard electrode potential (of 0.06 to 0.49 V) were used, and thus the PVP (having a weight-average molecular weight of 10000 to 40000) used as the polymer adsorbent was adsorbed onto the silver in a specific direction before the silver was precipitated, so that the growth of the silver in the specific direction was inhibited. Thus, it is considered that, in Examples 1 to 4, the silver was anisotropically grown, and the growth was promoted to generate the plate-shaped silver nanoparticles (see FIG. 3A to FIG. 3D).

In Comparative Example 1, however, ethylene glycol generally used in a polyol reduction method was used, and hence, reducing power for silver was higher than that in Examples 1 to 4. Therefore, it is considered that, even when the PVP used as the polymer adsorbent was mixed in the mixed liquid, precipitation of the silver proceeded before the polymer adsorbent was adsorbed onto the silver, resulting in generation of the spherical silver nanoparticles (see FIG. 4A). Therefore, it is considered that, if a reducing agent having too strong reducing power, such as ethylene glycol in Comparative Example 1, is used, plate-shaped silver nanoparticles cannot be obtained.

A citric acid was used as the polymer adsorbent, in place of the PVP, in Comparative Examples 2 and 3. The citric acid has a lower weight-average molecular weight than that of the PVP. Therefore, it is considered that the citric acid fails to be adsorbed onto the silver in a specific direction, and fails to protect the periphery of the silver. As a result, it is considered that the spherical or polyhedral silver nanoparticles were generated in Comparative Examples 2 and 3 (see FIG. 4B and FIG. 4C).

The PVP used as the polymer adsorbent in Comparative Example 4 has a higher weight-average molecular weight than that used in Examples 1 to 4. Therefore, it is considered that the polymer adsorbent cannot be adsorbed onto the periphery of a silver particle in an appropriate direction. As a result, it is considered that the polymer adsorbent agglomerated and the spherical or polyhedral silver nanoparticles were generated in Comparative Example 4 (see FIG. 4D).

Although the example embodiment of the disclosure has been described in detail, the specific configurations are not limited to the foregoing embodiment and examples. Modifications made without departing from the technical scope of the disclosure are included in the scope of the disclosure.

What is claimed is:

1. A method of producing plate-shaped silver nanoparticles, the method comprising:
preparing a mixed liquid by adding, to a solution containing silver ions, a reducing agent having a standard electrode potential within a range from 0.03 V to 0.8 V and polyvinylpyrrolidone having a weight-average molecular weight of 10000 to 40000; and
precipitating silver from the silver ions by irradiating the mixed liquid with a microwave.

2. The method according to claim 1, wherein the reducing agent is at least one of a citric acid, formalin, an ascorbic acid, an oxalic acid, and hydrogen peroxide.
